(19) United States
(12) Patent Application Publication
(10) Pub. No.: US 2025/0000425 A1
(43) Pub. Date: Jan. 2, 2025

(54) MULTI-SOURCE DOMAIN ADAPTIVE ELECTROENCEPHALOGRAM (EEG) EMOTIONAL STATE CLASSIFICATION METHOD BASED ON KNOWLEDGE DISTILLATION

(71) Applicant: Hangzhou Dianzi University, Hangzhou City (CN)

(72) Inventors: Hong ZENG, Hangzhou City (CN); Shuping LE, Hangzhou City (CN); Haohao ZHENG, Hangzhou City (CN); Deng PAN, Hangzhou City (CN); Tao ZENG, Hangzhou City (CN); Feifan XU, Hangzhou City (CN); Yu OUYANG, Hangzhou City (CN); Zhe JIA, Hangzhou City (CN); Dongguan QIAN, Hangzhou City (CN)

(21) Appl. No.: 18/646,512

(22) Filed: Apr. 25, 2024

(30) Foreign Application Priority Data
Jun. 30, 2023 (CN) 202310802378.2

(51) Int. Cl. A61B 5/369; A61B 5/00; A61B 5/16; G16H 50/50

(52) U.S. Cl. CPC A61B 5/369; A61B 5/165; A61B 5/7267; G16H 50/50

(57) ABSTRACT

A multi-source domain adaptive electroencephalogram (EEG) emotional state classification method based on knowledge distillation are provided. First, data is obtained for band-pass filtering, and artifacts are removed by an independent component analysis technology. Second, EEG features are extracted by using a differential entropy method, and a three-dimensional EEG time series is converted into a two-dimensional sample matrix. Then, a training set and a test set are defined in two task scenarios, respectively. A pseudo-label triple loss based on marginal sampling is combined with a maximum mean discrepancy. Knowledge is learned from different source domains, so that a plurality of single-source models are utilized to the greatest extent, and a more powerful model is implemented with less time consumption. Finally, the classification accuracy is used to evaluate the performance of the model in the two task scenarios.

### CROSS-REFERENCE TO RELATED APPLICATION

This patent application claims the benefit of and priority to Chinese Patent Application No. 202310802378.2 filed with the Chinese Patent Office on Jun. 30, 2023, which is hereby incorporated by reference herein in its entirety.

### TECHNICAL FIELD

The present disclosure belongs to the field of emotional state recognition of electroencephalogram (EEG) in the field of biometric recognition, and particularly relates to a multi-source domain adaptive EEG emotional state classification method based on knowledge distillation.

### BACKGROUND

Emotion recognition plays an important role in human-computer interaction. In recent years, with the improvement of computing power, emotion recognition methods based on deep learning have attracted more and more attention. These methods make decisions that reflect human emotions by deeply excavating the potential objective emotional features of users.

Affective brain-computer interfaces (aBCIs) are an important application of emotion recognition. By measuring the signals of the peripheral and central nervous system, features related to the emotional state of users are extracted, and these features are used to adjust human-computer interaction (HCI). The aBCIs show potential in rehabilitation and communication.

Generally speaking, emotion recognition mainly be classified into two categories: a method based on non-physiological signals, such as facial expression images, body gestures, and voice signals; and a method based on physiological signals, such as electroencephalography (EEG), electromyography (EMG) and electrocardiogram (ECG). However, compared with non-physiological signals, physiological signals can directly touch the internal emotional state of individuals, making the internal emotional state of individuals less susceptible to conscious or unconscious manipulation. Among various emotion recognition methods based on physiological signals, the EEG is one of the most commonly used methods, because the EEG is collected directly from the cerebral cortex and is very valuable for reflecting the psychological state of people. With the rapid development of the EEG collecting technology and the processing methods, emotion recognition based on the EEG has attracted more and more attention in recent years.

However, due to the low signal-to-noise ratio (SNR) and the significant individual differences between different subjects at different times, it is still a huge challenge to construct an efficient and robust emotion recognition deep learning model based on the EEG. In addition, it is very important to use the existing labeled data to analyze new unlabeled data in the brain-computer interface (BCI) based on the EEG. Therefore, domain adaptation is widely used in research work. By learning from the source data distribution, a model that performs well in related but different target data distribution is trained. However, in practice, there is usually a plurality of source domains, so that multi-source domain adaptation becomes a powerful extension of domain adaptation. Nevertheless, the technique used for domain alignment in multi-source domain adaptation is usually Maximum mean discrepancy (MMD), which only takes into account the adaptation of the domain level, but lacks the adaptation of the data pair level. Such limitation may lead to a lack of discriminative ability. In addition, in most multi-source domain adaptation frameworks, only the average prediction results of a plurality of single-source domain models are used as the final results, and these single-source domain models are not fully utilized.

### SUMMARY

In order to solve the defects of the prior art and make better use of the advantages of a plurality of single-source models, the present disclosure proposes a multi-source domain adaptive EEG emotional state classification method based on knowledge distillation (MS-KTF).

The technical scheme used by the present disclosure is as follows.

According to the present disclosure, the differential entropy (DE) features are used as frequency domain features of used EEG signals, the EEGNet model is slightly modified as the feature extractor, and a single-layer linear layer is used as a classifier to analyze the EEG signals, so as to implement a task of emotional state recognition in a cross-subject scenario and a cross-session scenario

According to the present disclosure, the training process is divided into three steps: (1) pre-training each teacher model based on each labeled source domain; (2) based on the corresponding labeled source domain and the unlabeled target domain, performing domain adaptation for each teacher model by using a source domain classification loss (SCL), a target domain classification loss (TCL), a maximum mean discrepancy (MMD) and a pseudo-label triplet loss; (3) transferring knowledge of teachers from a plurality of single-source domains to a student model. In addition, in step (2), in order to improve the effectiveness of the pseudo-label triplet loss, a margin-based sampling strategy is used to filter the original features, and only those features whose marginal scores are higher than the preset threshold are selected as embedded features for calculating the pseudo-label triplet loss.

The embodiment of that present disclosure includes the following steps.

In Step S1, data processing is performed.

The emotional data set SEED is taken as an example for analysis, and the processing steps of the original EEG data collected by an EEG collection device are as follows.

In Step S1-1, data denoising is performed.

The data set used by the present disclosure to verify the model performance comes from SEED. First, the original EEG signal collected in the data set is down-sampled to 200 HZ, then the band-pass filtering of 0-75 HZ is performed, ocular artifacts in the signals are removed by an independent component analysis (ICA) technology, and finally the traditional moving average and linear dynamic system (LDS) methods are used to further smooth the features.

In Step S1-2, differential entropy (DE) feature extraction is performed.

DE features are extracted from the EEG data after removing artifacts, and the data is segmented with a 1 s non-overlapping sliding window for each subject to obtain 3394 data samples. For each data sample xᵢ, the number of EEG data collecting channels is 62; and the frequency domain features of five frequency bands including δ(1-3 HZ), θ(4-7 HZ), α(8-13 HZ), β(14-30 HZ) and γ(31-50 HZ) are extracted.

In Step S2, data definitions and data set divisions are performed.

There are two test scenarios for emotional state classification in the method: the cross-subject scenario and the cross-session scenario, and the model tests in the two scenarios have their own different data definitions and data set divisions, which are explained in detail hereinafter.

It is assumed that there are N subjects, and each subject has D different session (period) tests. The whole sample set is expressed as U={(Xᵢ,Yᵢ)ᵢ₌₁^{N}}ⱼ₌₁^{D}, where i indicates a serial number of the subject, j indicates a serial number of a session (period), Xᵢ indicates a sample set of the subject i, and the corresponding label set is Yᵢ.

For a task of emotional state classification in the cross-session scenario, the data set is cross-verified by using a leave-one-out method; specifically, in each subject i, the data of 15 emotional tests of all subjects in a latest session is taken as a test set; for remaining D−1 sessions, in a unit of session, each session is deem as a source domain in a training set, and finally, D−1 source domains are obtained as the training set; and a total of N tests are conducted and the average accuracy is calculated.

For a task of emotional state classification in the cross-subject scenario, the data set is cross-verified by using the leave-one-out method; specifically, in a session (period), the data of all 15 emotional tests of a subject are iteratively extracted with an assumption that an emotional state label thereof is unknown as a test set; from remaining N−1 subjects, R subjects are randomly and unrepeatably grouped into a group, as a source domain in the training set; finally

$$⌊ \frac{N - 1}{R} ⌋$$

(round down) source domains are obtained as the training set, a total of D×N tests are conducted, and the average accuracy is calculated.

In Step S3, the construction and training of the MS-KTF model is performed.

The main parameters in the neural network MS-KTF model include:
(1) a dimension d_{f} of the feature embedding space, that is, a dimension that features enter the embedding space after being extracted by the feature extractor, which is closely related to a characterization ability of embedded features;
(2) the threshold for margin-based sampling, that is, the key parameter to determine whether the feature should be sampled, which samples the feature with a score higher than the threshold;
(3) the temperature for distillation learning, that is, a temperature coefficient in softmax operation, which is mainly used to adjust the smoothness of the predicted distribution condition. In Step S3-1, initialization is performed.

The MS-KFT model consists of two parts: teacher models, which each are based on a corresponding single source domain, and a student model acting on the target domain, both the teacher models and the student model each consist of two modules: a domain-specific feature extractor N_{f} and a label classifier N_{y}; a plurality of single-source domain teacher models based on the multi-source domain and parameters of a target domain student are initialized.

In Step S3-2, a plurality of single-source domain teacher models are pre-trained.

Based on a multi-source domain sample set, a feature extractor N_{f} and a label classifier N_{y} of each domain-specific teacher model are pre-trained using a corresponding labeled single source domain sample set, such that each domain-specific teacher model has a certain pattern recognition ability in its respective source domains.

In Step S3-3, domain adaptation is performed on feature extractors of a plurality of single-source domain teacher models.

One labeled source domain sample set and one unlabeled target domain sample set are formed into a branch. In each branch, the feature extractor N_{f} corresponding to the domain-specific teacher model is used to extract features from the respective source domain sample and target domain sample, and the features are extracted from the original feature space into the embedded space.

Thereafter, the embedded features are aligned at a domain level based on the maximum mean discrepancy in the feature space; the embedded features are aligned at a data pair level based on the pseudo-label triplet loss of margin-based sampling.

By minimizing the maximum mean discrepancy and the pseudo-label triplet loss, the feature extractors N_{f} of a plurality of single-source domain teacher models are trained to extract domain-invariant features in the source domain and the target domain.

In Step S3-4, label classifiers N_{y} of a plurality of single-source domain teacher models are trained.

In each single-source domain teacher model, the extracted source domain feature information is passed through the label classifier N_{y} to obtain predicted emotion Ŷ^{S}, and a cross-entropy between the predicted emotion Ŷ^{S} and a corresponding label Y^{S} in an actual sample is calculated; similarly, a cross-entropy between a predicted emotion Ŷ^{T} of the target domain feature information and a generated pseudo-label Ŷ^{T} is calculated.

By minimizing the two obtained cross-entropies, the label classifiers N_{y} of a plurality of single-source domain teacher models are trained to have good emotion classification ability in their respective source domain and respective target domain.

In Step S3-5, the knowledge of a plurality of single-source domain teacher models is merged.

Two different merging strategies are used to balance performances of the teacher models.
(1) The teacher models to be merged is screened based on a voting manner.

This strategy is more suitable for a case in which teacher models have poor performance balance. Based on an emotion prediction result Ŷ_{teacher}^{T} obtained by the feature extractor and the label classifier of each teacher model, with a unlabeled target domain sample, a corresponding one-hot coding result Ô_{teacher}^{T} is generated; voting is performed based on the one-hot coding result Ô_{teacher}^{T} generated by each teacher model, and the voting result is deem as a decision variable \hat{D}^{T}; in a case that the emotion prediction result Ŷ_{teacher}^{T} of the teacher model is the same as the decision variable \hat{D}^{T}, the teacher model is selected for knowledge merging.

A mean value Y̅_{teacher}^{T} of emotion prediction results of all selected teacher models is calculated as the merged knowledge of all the teacher models.
(2) The teacher models are merged in an average manner.

This strategy is more suitable for a case in which teacher models have strong performance balance; in this case, all the teacher models have a same weight, and the mean value Y̅_{teacher}^{T} of the prediction results of all the selected teacher models is calculated as the merged knowledge of all the teacher models.

In Step S3-6, the merged knowledge of the teacher models is taught to the student model.

With unlabeled target domain sample data, a prediction result Ŷ_{student}^{T} of the student model is obtained through the feature extractor and the label classifier of the student model;

based on a predetermined distillation temperature, smoothing processing is performed on the merged knowledge Y̅_{teacher}^{T} of the teacher model and the prediction result Ŷ_{student}^{T} of the student model; and Kullback-Leibler (KL) divergence is used to evaluate a difference between two prediction results;

by minimizing the KL divergence between the teacher models and the student model so that the student model learns the knowledge of the teacher models, more extensive feature extraction and label classification ability than the teacher models in the target domain are obtained.

In Step S4, model performance evaluation in two scenarios including a cross-session scenario and a cross-subject scenario is performed.

The present disclosure specifically verifies the performance of the model on the SEED data set.

The emotional state Ŷ_{student}^{T} predicted by the target domain sample set in the training convergent student model is compared with the real state Y^{T}, the accuracy result is obtained, and the model performance is evaluated. The accuracy rate is a number of correctly classified samples during model testing to a total number of test samples, and the calculation formula of the model accuracy is as follows:

$$a c c = \frac{T P + T N}{T P + F P + F N + T N} . \qquad ( 1 )$$

Where TP is a positive sample of a positive type predicted by the model, TN is a negative sample of a negative type predicted by the model, FP is a negative sample of a positive type predicted by the model, and FN is a positive sample of a negative type predicted by the model.

A multi-source domain adaptive EEG emotional state classification system based on knowledge distillation includes a pre-training module, a teacher model domain adaptation module and a student model training module; where the pre-training module pre-trains each teacher model based on each labeled source domain; the teacher model domain adaptation module performs domain adaptation for each teacher model by using a source domain classification loss (SCL), a target domain classification loss (TCL), a maximum mean discrepancy (MMD) and a pseudo-label triplet loss based on the corresponding labeled source domain and an unlabeled target domain; and the student model training module transfers knowledge of teachers from a plurality of single-source domains to a student model. The pre-training module, the teacher model domain adaptation module and the student model training module are computer implemented modules.

In addition, in the teacher model domain adaptation module, in order to improve effectiveness of the pseudo-label triplet loss, a margin-based sampling strategy is used to filter original features, and only features with marginal scores higher than a preset threshold are selected as embedded features for calculating the pseudo-label triplet loss.

The beneficial effect of that present disclosure are as follows.

The present disclosure solves the blind estimation problem of the maximum mean discrepancy (MMD) technology in the multi-source domain adaptation by using the pseudo-label triplet loss. In addition, the margin-based sampling strategy based on uncertainty measurement is used to improve its effectiveness, while knowledge extraction technology is introduced to train a more robust student model by teaching the knowledge of a plurality of teacher models, so as to make maximum use of multi-source domain knowledge. Through the experimental verification on the public emotion data set SEED, compared with the previous method, the present disclosure has achieved significant improvement.

### BRIEF DESCRIPTION OF THE DRAWINGS

FIG. 1 is a schematic flow diagram of the present disclosure.

FIG. 2 is a detailed structural diagram of a feature extractor of a model according to the present disclosure.

FIG. 3 is an architecture diagram of an MS-KTF model.

FIG. 4 is a diagram of data division and construction of the MS-KTF model.

FIG. 5 is a structural diagram of a teacher model.

FIG. 6 is a structural diagram of a student model.

### DETAILED DESCRIPTION OF THE EMBODIMENTS

The preferred embodiments of the present disclosure will be described in detail with reference to the attached drawings hereinafter, so that the advantages and features of the present disclosure can be more easily understood by those skilled in the art, and the scope of protection of the present disclosure can be more clearly defined.

Multi-source domain adaptation (MSDA) aims to transfer knowledge from a plurality of source domains to an unlabeled target domain, which is very suitable for cross-session and cross-subject EEG emotion recognition. However, the existing MSDA model only takes into account the domain level of each pair of feature relationships between the source domain and the target domain, but rarely takes into account the correlation of the data pair level between the two domains, resulting in poor robustness.

The present disclosure discloses a multi-source domain knowledge transfer framework (MS-KTF) for EEG emotional recognition. First, data is obtained for band-pass filtering, and artifacts are removed by an independent component analysis (ICA) technology. Second, EEG features are extracted by using a differential entropy (DE) method, and a three-dimensional EEG time series is converted into a two-dimensional sample matrix. Then, a training set and a test set are defined in two task scenarios, respectively, so as to ensure that the training set and the test set are not overlapped with each other. For these samples, MS-KTF combines a pseudo-label triple loss based on margin-based sampling with a maximum mean discrepancy (MMD). According to the method, unbiased alignment between each pair of source domains and target domains can be implemented at the domain level and the data pair level. Specifically, the framework learns knowledge from different source domains, so that a plurality of single-source models are utilized to the greatest extent, and a more powerful model is implemented with less time consumption. Finally, the classification accuracy is used to evaluate the performance of the model in the two task scenarios. According to the present disclosure, the triple loss and the maximum mean discrepancy are combined, so that the problem of insufficient alignment of EEG signal distribution differences is solved to a certain extent, and a high-precision cross-session and cross-subject emotional state classifier is trained, which has the advantages of low time complexity, high calculation efficiency, strong generalization ability and the like, so as to have a wide application prospect in the actual brain-computer interaction.

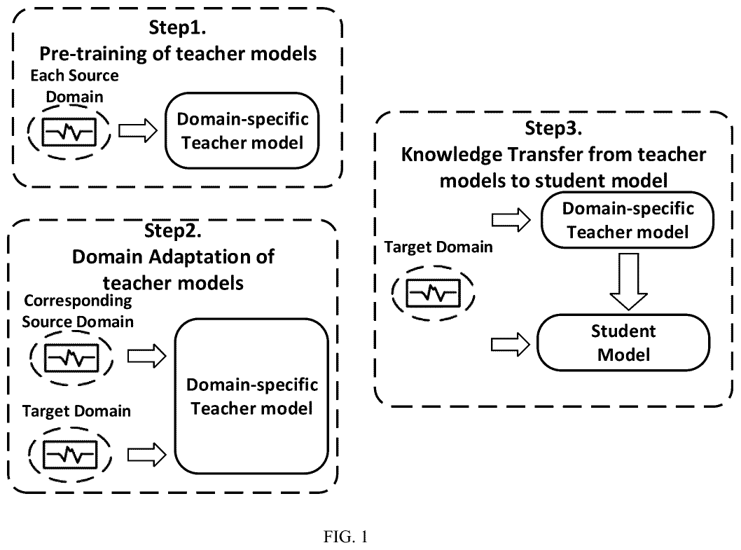

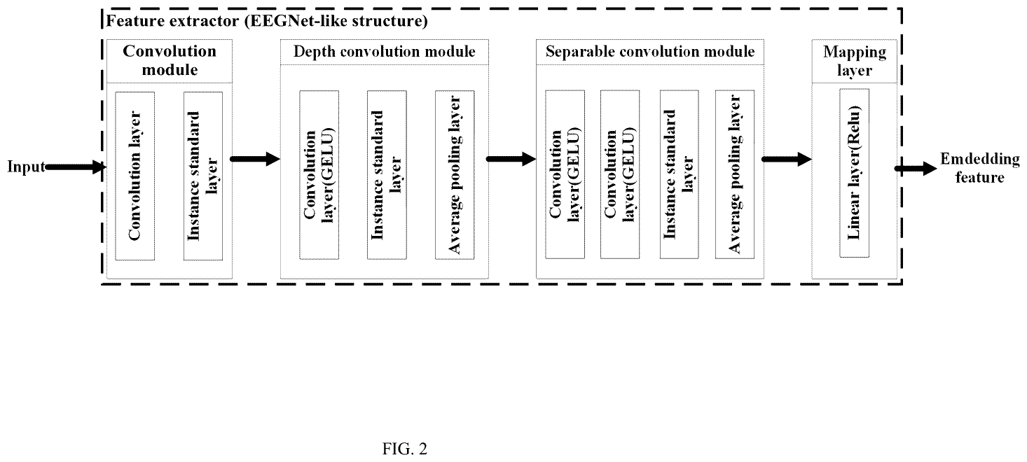

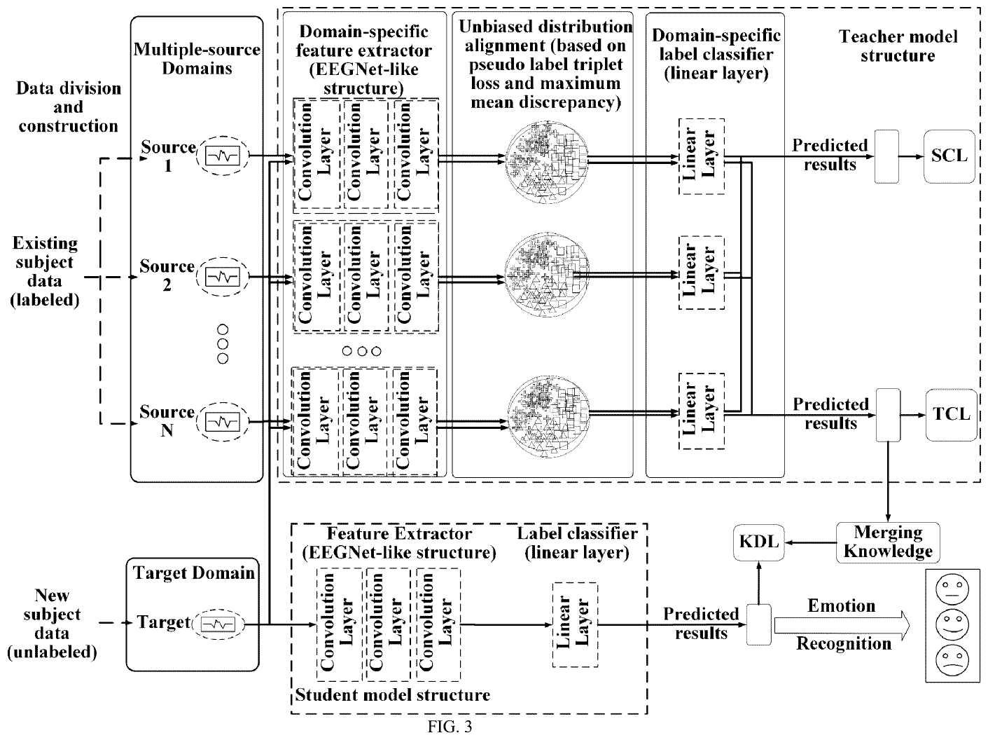

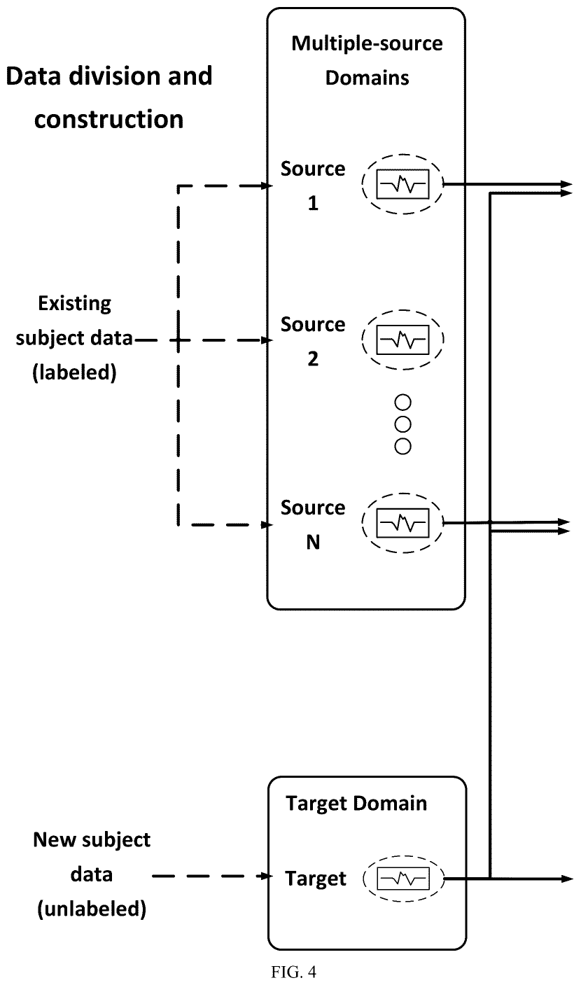

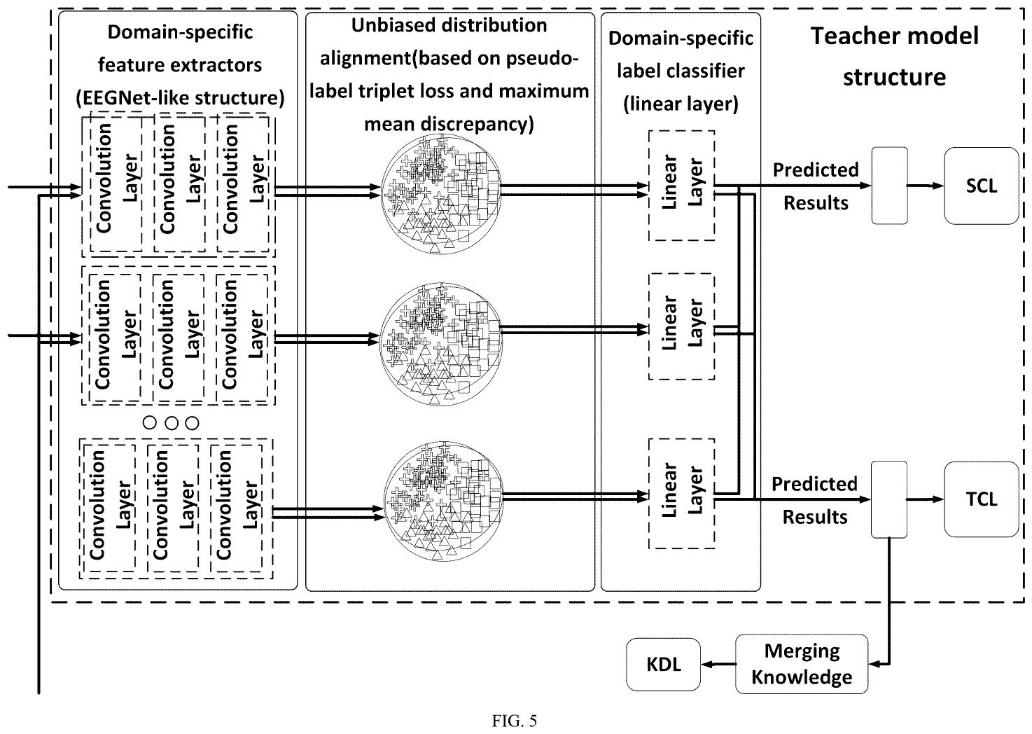

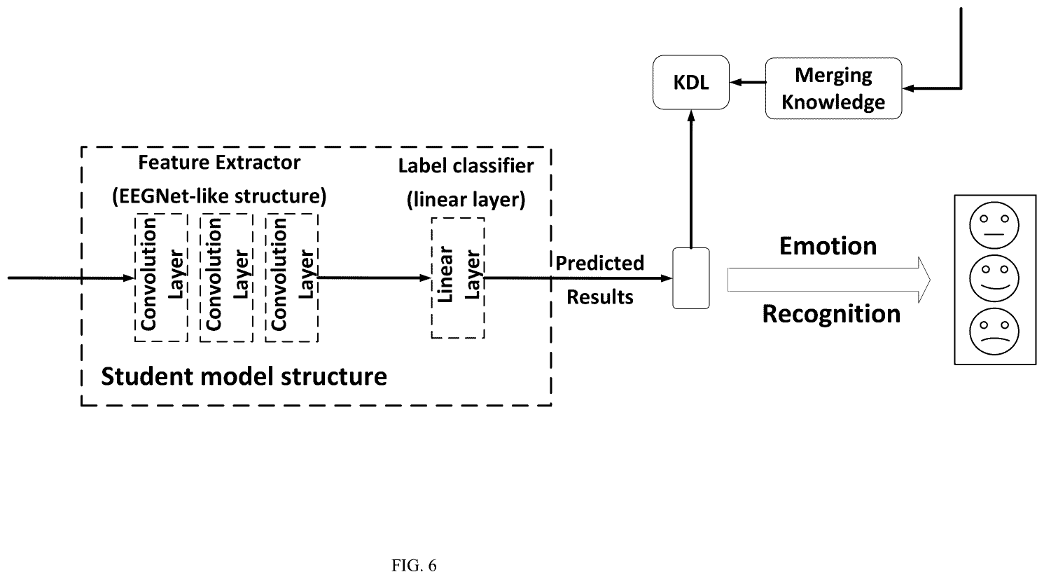

Refer to FIG. 1, FIG. 2, FIG. 3, FIG. 4, FIG. 5 and FIG. 6 for the specific implementation of the present disclosure. The embodiment of the present disclosure includes the following steps.

In Step S1, data processing is performed.

The emotional data set is taken as an example for analysis, and the processing steps of the original EEG data collected by an EEG collection device are as follows.

In Step S1-1, data denoising is performed.

The data set used by the present disclosure to verify the model performance comes from SEED. Refer to the paper “Investigating Critical Frequency Bands and Channels for EEG-Based Emotion Recognition with Deep Neural Networks” for details. First, the original EEG signal collected in the data set is down-sampled to 200 HZ, then the band-pass filtering of 0.3-50 HZ is performed, and finally ocular artifacts in the signals are removed by an independent component analysis (ICA) technology.

In Step S1-2, differential entropy (DE) feature extraction is performed.

DE features are extracted from the EEG data after removing artifacts. Each subject will watch 15 videos that can result in obvious emotional changes of the subject, and the EEG data collected within the same video playing duration is regarded as an emotional test. Each subject has 15 emotional tests. The data is segmented with a 1 s non-overlapping sliding window for each subject to obtain 3394 data samples. For each data sample xᵢ, the number of EEG data collecting channels is 62; and the frequency domain features of five frequency bands including: δ(1-3 HZ), θ(4-7 HZ), α(8-13 HZ), β(14-30 HZ) and γ(31-50 HZ) are extracted.

In Step S2, data definitions and data set divisions are performed.

There are two test scenarios for emotional state classification: the cross-subject scenario and the cross-session scenario, and the model tests in the two scenarios have their own different data definitions and data set divisions, which are explained in detail hereinafter.

It is assumed that there are N subjects, and each subject has D different (session) period tests. The whole sample set is expressed as U={(Xᵢ,Yᵢ)ⱼ₌₁^{D}}ᵢ₌₁^{N}, where i indicates a serial number of the subject, j indicates a serial number of a session (period), Xᵢ indicates a sample set of the subject i, and a corresponding label set is Yᵢ.

For a task of emotional state classification in the cross-session scenario, the data set is cross-verified by using a leave-one-out method; specifically, in each subject i, the data of 15 emotional tests of all subjects in a latest session is taken as a test set; for remaining D−1 sessions, in a unit of session, each session is deemed as a source domain in a training set, and finally D−1 source domains are obtained as a training set; a total of N tests are conducted and the average accuracy is calculated.

For a task of emotional state classification in the cross-subject scenario, the data set is cross-verified by using the leave-one-out method; specifically, in a session (period), the data of all 15 emotional tests of a subject are iteratively extracted with an assumption that an emotional state label thereof is unknown, as a test set; from remaining N−1 subjects, R subjects are randomly and unrepeatably grouped into a group, as a source domain in the training set; and

$$⌊ \frac{N - 1}{R} ⌋$$

(round down) source domains are obtained as the training set. Finally, the performance of the model is verified on the test set of N subjects, a total of D×N tests are conducted, and the average accuracy is calculated.

In Step S3, the construction and training of the MS-KTF model is performed.

The main parameters in the neural network MS-KTF model include:
(1) a dimension d_{f} of the feature embedding space, that is, a dimension that features enter the embedding space after being extracted by the feature extractor, which is closely related to a characterization ability of embedded features;
(2) the threshold for margin-based sampling, that is, the key parameter to determine whether the feature should be sampled, which samples the feature with a score higher than the threshold;
(3) the temperature for distillation learning, that is, a temperature coefficient in softmax operation, which is mainly used to adjust the smoothness of the predicted distribution condition. In Step S3-1, Specific data division and input of the model is performed.

In Step S3-1-1, the data division of the model is performed.

The data division and construction of the model are shown in FIG. 4, and the specific division condition is described as follows:

for the cross-subject scenario: a target domain sample set of the model is U^{T}={Xᵢ}, where Xᵢ indicates the feature data set of an i-th subject; Xᵢ={xⱼ}ⱼ₌₁ⁿ; xⱼ indicates a j-th sample in Xᵢ, and n indicates a number of samples in Xᵢ; the multi-source domain sample set of the model is

$${U}^{s} = {\{ {( {X}_{i} , {Y}_{i} )}_{i ∈ {P}_{j}} \}}_{j = 1}^{⌊ \frac{N - 1}{R} ⌋} ,$$

Pⱼ⊆[N]\i, Pⱼ∩Pₖ=Ø, ∀i∀k, i≠j, where [N]\i indicates a serial number set of all the subjects after the i-th subject data is removed, and Pⱼ indicates a serial number set of the subjects included in the j-th source domain (* all the data in the cross-subject scenario comes from the same session).

For the cross-session scenario: the target domain sample set of the model is U^{T}={(Xᵢ)ᵢ₌₁^{N}}ⱼ, where Xᵢ indicates a feature data set of an i-th subject; j indicates a j-th session (period); the multi-source domain sample set of the model is U^{S}={(Xᵢ,Yᵢ)ᵢ₌₁^{N}}ₖ, k∈[D]/j, where [D]/j indicates the a serial number set of all the sessions after the j-th session is removed.

In Step S3-1-2, data input of the model is performed.

As shown in the left half of FIG. 3, that is, as show in FIG. 4, one labeled source domain sample set U^{S} and one unlabeled target domain sample set U^{T} are formed into a branch for a subsequent training of each single-source domain teacher model; and for the student model, only the unlabeled target domain sample set U^{T} is used.

In Step S3-2, initialization of the model is performed.

As shown in the right half of FIG. 3, the MS-KFT model consists of two parts: teacher models, which each are based on a corresponding single source domain (the upper right corner of FIG. 3), and a student model only acting on the target domain (the lower right corner of FIG. 3). Both the teacher models and the student model each consist of two modules: a domain-specific feature extractor N_{f} and a label classifier N_{y}. The specific structure of the feature extractor N_{f} is shown in FIG. 2, and the label classifier N_{y} consists of a single-layer linear layer and a softmax function.

In Step S3-3, pre-training single-source domain teacher models are performed.

As shown in FIG. 5, it is a structural diagram of a plurality of single-source domain teacher models. Each of the domain-specific feature extractor N_{f} and the domain-specific label classifier N_{y} forms a single-source domain teacher model.

Based on a multi-source domain sample set U^{S}, a feature extractor N_{f} and a label classifier N_{y} of each domain-specific teacher model are pre-trained using a corresponding labeled single source domain sample set, such that each domain-specific teacher model has a certain pattern recognition ability in its respective source domains (the optimization goal is the same as SCL in the following formula (5), which will not be described in detail here).

In Step S3-4, training of the feature extractor of single-source domain teacher models is performed.

After passing through the feature extractor N_{f} of the domain-specific teacher model, the respective low-dimensional features F^{S} and F^{T} of the corresponding source domain data U^{S} and the target domain data U^{T} are extracted. In order to ensure the unbiased adaptability of the extracted features, two methods including domain-level distribution alignment and data-pair-level distribution alignment are used in this patent.

In Step S3-4-1, domain-level distribution alignment is performed.

Corresponding to the unbiased distribution alignment in FIG. 5, based on the pseudo-label triple loss and the maximum mean discrepancy, the domain adaptation of each teacher model is performed in this patent.

The maximum mean discrepancy (MMD) is a distance metric in probabilistic metric space, which is widely used in machine learning and nonparametric testing. The distance metric is based on the idea of embedding the probability into reproducing kernel Hilbert space (RKHS), which aims to reduce the distribution difference between the source domain and the target domain while retaining their specific discriminant information. In the training process, the distance between the source domain and the target domain in the feature space is reduced by minimizing the maximum mean discrepancy (MMD) loss, so as to achieve domain-level alignment, and a specific formula is as follows:

$$M M {D}_{( {F}^{S} , {F}^{T} )} = {‖ \frac{1}{{N}^{S}} {\sum }_{i = 1}^{{N}^{S}} {F}_{i}^{S} - \frac{1}{{N}^{T}} {\sum }_{j = I}^{{N}^{T}} {F}_{j}^{T} ‖}_{ℋ}^{2} . \qquad ( 2 )$$

Where Fᵢ^{S} and Fⱼ^{T} indicate the extracted low-dimensional features of the i-th sample in the source domain and the j-th sample in the target domain, respectively; N^{S} and N^{T} indicate a number of source domain samples and a number of target domain samples respectively.

In Step S3-4-2: data-pair-level distribution alignment is performed.

Because the MMD blindly estimates parameters to take into account statistical information and their relationships, feature distinguishability may be reduced, and the relationship between the intra-class distance and the inter-class distance may be affected, because one of the distance values decreases and the other distance value increases. The triple loss can reduce the intra-class distance and increase the inter-class distance, which is a way to solve this problem. However, in domain adaptation, the target domain is usually unlabeled. Therefore, the triple loss of margin-based sampling is used to perform data-pair-level distribution alignment in this patent.

A margin-based score of the prediction result of each sample is used in this patent as a basis for determining whether the sample is sampled, and this method may be expressed by the following formula:

$$margin ( x ) = {{g}_{θ} ( {F}^{T} )}_{{i}^{*}} - {max}_{i ∈ [ k ] ∖ {i}^{*}} \{ {{g}_{θ} ( {F}^{T} )}_{i} \} {X}_{s e lected} = \{ {x}_{j} \vert margin ( {x}_{j} ) \geq Threshold , {x}_{j} ∈ X \} . \qquad ( 3 )$$

Where x is an input sample, g_{θ} is an abstract function of the label classifier, i* is a category with a highest prediction probability in the prediction result, k is a number of all categories, [k]\i* indicates a set of all the categories except i*, and Threshold is a predetermined threshold of margin-based sampling.

The triplet loss requires sampling in a form of triplet {xᵢ^{a},xᵢ^{p},xᵢⁿ}ᵢ^{N}^{ₜᵣᵢₚ}, in which xᵢ^{a} (an anchor sample) and xᵢ^{p} (a positive sample) are different samples of the same category from the i-th triplet, xᵢⁿ is any sample of the i-th triplet of a category different from that of anchor sample xᵢ^{a}; the purpose of the triple loss is to ensure that a distance between the embedded features of a positive sample pair (xᵢ^{a},xᵢ^{p}) plus a fixed margin value is smaller than a distance between the embedded features of a negative sample pair (xᵢ^{a},xᵢⁿ). Formally, for a mini-batch sample set, the triple loss is defined as:

$${d}_{p} ( {x}_{i}^{a} , {x}_{i}^{p} ) = ‖ {f}_{θ} ( {x}_{i}^{a} ) - {f}_{θ} ( {x}_{i}^{p} ) ‖ \\ {d}_{n} ( {x}_{i}^{a} , {x}_{i}^{n} ) = ‖ {f}_{θ} ( {x}_{i}^{a} ) - {f}_{θ} ( {x}_{i}^{n} ) ‖ {ℒ}_{trip} = \frac{1}{N} {\sum }_{{x}_{i} ∈ {X}_{s e l e c t e d}} max \{ {d}_{p} ( {x}_{i}^{a} , {x}_{i}^{p} ) - {d}_{n} ( {x}_{i}^{a} , {x}_{i}^{n} ) + α , 0 \} . \qquad ( 4 )$$

Where N is a number of samples contained in X_{selected}, α is a predetermined margin value for guiding separability, d(⋅) is a function for calculating an Euler distance between regularized embedded feature pairs, and f_{θ}(⋅) is an abstract function for feature extraction.

In Step S3-5, training of label classifiers of single-source domain teacher models is performed.

The cross-entropy (CE) loss is used as an evaluation index of the classification result of the label classifier in the source domain and the target domain in the patent, a source classification loss (SCL) is specifically used as the classification loss in the source domain, and a target classification loss (TCL) is specifically used as the classification loss in the target domain.

In the source domain, there is a real label, so the SCL uses the real label and the classification result of the label classifier as the comparison object, and the specific formula is as follows:

$${w}_{i} = \{ \begin{matrix}1 , if {\hat{y}}_{i}^{s} = {y}_{i}^{s} \\ 0 , otherwise\end{matrix} {ℒ}_{S C L} = - \sum {w}_{i} ln ( {g}_{θ} ( {f}_{θ} ( {x}_{i} ) ) ) . \qquad ( 5 )$$

Where xᵢ is an i-th source domain input sample, yᵢ^{S} is a real label of the i-th source domain input sample, ŷᵢ^{S} is a prediction result of the label classifier for the i-th source domain input sample, f_{θ}(⋅) is an abstract function for feature extraction, and g_{θ}(⋅) is an abstract function of the label classifier.

In the target domain, the sample lacks a real label, and the corresponding TCL uses the generated pseudo label and the classification result of the label classifier as comparison objects, and a specific formula is as follows:

$${\tilde{y}}_{i}^{T} = arg max ( {g}_{θ} ( {f}_{θ} ( {x}_{i} ) ) ) , {w}_{i} = \{ \begin{matrix}1 , if {\hat{y}}_{i}^{T} = {\tilde{y}}_{i}^{T} \\ 0 , otherwise\end{matrix} , {ℒ}_{T C L} = - \sum {w}_{i} ln ( {g}_{θ} ( {f}_{θ} ( {x}_{i} ) ) ) , \qquad ( 6 ) .$$

Where xᵢ is an i-th target domain input sample, \tilde{y}ᵢ^{T} is a pseudo label generated by the i-th target domain input sample, ŷᵢ^{T} is a prediction result of the label classifier for the i-th target domain input sample, f_{θ}(⋅) is an abstract function for feature extraction, and g_{θ}(⋅) is an abstract function of the label classifier.

In Step S3-6, the goal optimization and the training of the single-source domain teacher model is performed.

Step S3-4 and Step S3-5 are summarized, in a domain adaptation stage of the teacher model, the final optimization goal is shown in a following formula:

$${min}_{{f}_{θ} , {g}_{θ}} {ℒ}_{D} = {min}_{{f}_{θ} , {g}_{θ}} ( {ℒ}_{S C L} + {βℒ}_{M M D} + {γℒ}_{trip} + {σℒ}_{T C L} ) . \qquad ( 7 )$$

Where β, γ and σ are weighting factors for balancing a loss function.

By using a random gradient optimizer and combining with a mini-batch training mode, domain invariant features are obtained for each pair of source domain and target domains at the domain level and a data pair level through minimizing the MMD loss and the triple loss ( [symbol] _{MMD}, [symbol] ₜᵣᵢₚ) in formula (7); by minimizing the classification losses ( [symbol] _{SCL}, [symbol] _{TCL}) in the source domain and the target domain, a better classifier is obtained, which accurately predicts the source domain samples without sacrificing the ability to discriminate the target domain samples.

In Step S3-7, training of the student model is performed.

The structure of the specific student model can be seen in FIG. 6. After the domain adaptation of the teacher models, a plurality of single-source domain models are obtained. The models can effectively extract the deep EEG pattern representations that are differentiated for classification tasks but transferable between different domains. In order to maximize the utilization of these single-source models, knowledge distillation is used to transfer knowledge learned from the multi-source domain and train a more powerful student model.

In order to better merge the knowledge of the teacher models, a voting-based method is used to select the knowledge of the teacher models to be merged, which is expressed as the following formula in the patent:

$$\{ {\tilde{y}}_{i} \} = {mode}_{j = 1}^{{N}_{t}} ( {\hat{y}}_{i}^{{T}_{j}} ) , {mask}_{i}^{{T}_{j}} = \{ \begin{matrix}0 , if {\hat{y}}_{i}^{{T}_{j}} ∉ \{ {\tilde{y}}_{i} \} \\ 1 , otherwise\end{matrix} , {merge}_{i} = a v {g}_{j = 1}^{{N}_{t}} ( {g}_{θ}^{{T}_{j}} ( {f}_{θ}^{{T}_{j}} ( {x}_{i} ) ) * {mask}_{i}^{{T}_{j}} ) . \qquad ( 8 )$$

Where xᵢ is an i-th input sample, Nₜ is a number of teacher models, mode(⋅) is a function for finding a mode/multiple modes, * is a point multiplication function, ŷᵢ^{T}^{ⱼ} is a prediction result of a j-th teacher model for the i-th input sample, and {\tilde{y}ᵢ} is a decision label set for generating a teacher model mask of the i-th input sample.

After obtaining the merged knowledge of a plurality of single-source domain teacher models, Kullback-Leibler (KL) divergence is used to evaluate a difference between the prediction result of the teacher model and the prediction result of the student model, and the formula is as follows:

$${ℒ}_{K D} = KLD [ ( {g}_{θ} ( {f}_{θ} ( X ) ) ; T ) , ( merge ; T ) ] . \qquad ( 9 )$$

Where X is an input sample set, merge is a merged teacher knowledge set, T is a predetermined temperature coefficient for controlling the smoothness of softmax function, and KLD[p,q] is an evaluation function for measuring a KL divergence between a distribution p and a distribution q.

By using an adam optimizer and combining with a mini-batch training mode, a KL loss in formula (9) is minimized, so that the student model fully learns the merged knowledge of the teacher model and obtains better performance in the target domain.

In Step S4: model performance evaluation in two scenarios including a cross-session scenario and a cross-subject scenario is performed.

The present disclosure specifically verifies the performance of the model on the SEED data set and the SEED-IV data set.

The prediction result y_{pred} obtained by the converged student model in the target domain is compared with the real label y^{T} in the target domain by using a confusion matrix, and the comparison result is obtained to evaluate the model performance. The accuracy rate is a number of correctly classified samples during model testing to a total number of test samples, and the calculation formula of the model accuracy is as follows:

$$a c {c}_{s} = \frac{T P + T N}{T P + F P + F N + T N} , s = 1 , 2 , … , 45. \qquad ( 10 )$$

Where TP is a positive sample of a positive type predicted by the model, TN is a negative sample of a negative type predicted by the model, FP is a negative sample of a positive type predicted by the model, and FN is a positive sample of a negative type predicted by the model. SEED data includes 15 subjects, and each subject has three tests with a total of 45 tests. The average accuracy of the first two tests of 15 subjects is as follows:

$$a c {c}_{a l l} = \frac{\sum {acc}_{s}}{2 * 15} , s = 1 , 2 , … , 30. \qquad ( 11 )$$

The mean square error formula of the result is as follows:

$$s t {d}_{a l l} = \sqrt{\frac{\sum {( {acc}_{a l l} - a c {c}_{s} )}^{2}}{2 * 15}} , s = 1 , 2 , … , 30. \qquad ( 12 )$$

Refer to Step S3-1-1 for the data set division in the two scenarios including the cross-subject scenario and the cross-session scenario. For the cross-subject scenario, the model proposed by the present disclosure is tested on EEG data of 15 subjects in one test. For the cross-session scenario, the model proposed by the present disclosure is tested on EEG data of 15 subjects in one test. The comparison between the final test results and the existing technologies (SVM, DGCNN and RGNN) is shown in the following table:

TABLE 1
table of comparison of classifier performance on the
SEED data set

| | | | | the present |
|---|---|---|---|---|
| Classifier | DDC | DAN | MS-MDA | disclosure |
| accuracy rate of | 81.53/6.83 | 79.93/7.06 | 88.56/7.80 | 97.58/1.46 |
| cross-session | | | | |
| accuracy rate of | 68.99/3.23 | 65.84/2.25 | 89.63/6.79 | 91.73/10.48 |
| cross-subject | | | | |

TABLE 2
table of comparison of classifier performance on the
SEED-IV data set

| | | | | the present |
|---|---|---|---|---|
| Classifier | DDC | DAN | MS-MDA | disclosure |
| accuracy rate of | 57.63/11.28 | 55.14/12.79 | 61.43/15.71 | 77.70/13.49 |
| cross-session | | | | |
| accuracy rate of | 37.71/6.36 | 32.44/9.02 | 59.34/5.48 | 74.19/15.84 |
| cross-subject | | | | |

As can be seen from the results in the above tables, the method proposed by the present disclosure has higher accuracy than those of DDC, DAN and MS-MDA in the cross-session scenario and the cross-subject scenario. The present disclosure is not only suitable for the research of emotional state recognition, but also suitable for any EEG-based cross-session and cross-subject classification prediction task, which solves the problem of individual differences of the EEG to some extent.

What is claimed is:

1. A multi-source domain adaptive electroencephalogram (EEG) emotional state classification method based on knowledge distillation, which uses differential entropy features as frequency domain features of used EEG signals, an improved EEGNet model as a feature extractor, and a single-layer linear layer as classifier to analyze the EEG signals, so as to implement a task of emotional state recognition in a cross-subject scenario and a cross-session scenario, comprising:
I. pre-training each teacher model based on each labeled source domain;
II. based on the corresponding labeled source domain and an unlabeled target domain, performing domain adaptation for each teacher model by using a source domain classification loss, a target domain classification loss, a maximum mean discrepancy and a pseudo-label triplet loss; wherein in order to improve effectiveness of the pseudo-label triplet loss, a margin-based sampling strategy is used to filter original features, and only features with marginal scores higher than a preset threshold are selected as embedded features for calculating the pseudo-label triplet loss;
III. transferring knowledge of teachers from a plurality of single-source domains to a student model.

2. The multi-source domain adaptive EEG emotional state classification method based on knowledge distillation according to claim 1, wherein there are two test scenarios for emotional state classification in the method: the cross-subject scenario and the cross-session scenario, and model tests in the two scenarios have their own different data definitions and data set divisions, and the specific data definitions and data set divisions are as follows:
assuming that there are N subjects, and each subject has D different sessions; the whole sample set is expressed as U={(Xᵢ,Yᵢ)ᵢ₌₁^{N}}ⱼ₌₁^{D}, where i indicates a serial number of the subject, j indicates a serial number of a session, Xᵢ indicates a sample set of a subject i, and a corresponding label set is Yᵢ;
for a task of emotional state classification in the cross-session scenario, the data set is cross-verified by using a leave-one-out method; in each subject i, data of 15 emotional tests of all subjects in a latest session is taken as a test set; for remaining D−1 sessions, in a unit of session, each session is deemed as a source domain in a training set, and D−1 source domains are obtained as the training set; a total of N tests are conducted and an average accuracy is calculated;
for the task of emotional state classification in the cross-subject scenario, the data set is cross-verified by using the leave-one-out method; in a session, data of all 15 emotional tests of a subject are iteratively extracted with an assumption that an emotional state label thereof is unknown, as a test set; from remaining N−1 subjects, R subjects are randomly and unrepeatably grouped into a group, as a source domain in the training set;
$$⌊ \frac{N - 1}{R} ⌋$$
source domains are obtained as the training set, a total of D×N tests are conducted, and an average accuracy is calculated.

3. The multi-source domain adaptive EEG emotional state classification method based on knowledge distillation according to claim 1, wherein construction and training of a multi-source domain knowledge transfer framework (MS-KTF) model during implementation of the method are as follows:
S3-1: initialization;
the MS-KFT model consists of two parts: teacher models, which each are based on a corresponding single source domain, and a student model acting on a target domain. both the teacher models and the student model each consist of two modules: a domain-specific feature extractor N_{f} and a label classifier N_{y}; a plurality of single-source domain teacher models based on the multi-source domain and parameters of a target domain student are initialized;
S3-2: pre-training the plurality of single-source domain teacher models;
based on a multi-source domain sample set, a feature extractor N_{f} and a label classifier N_{y} of each domain-specific teacher model are pre-trained using a corresponding labeled single-source domain sample set, such that each domain-specific teacher model has a certain pattern recognition ability in its respective source domain;
S3-3: performing domain adaptation on feature extractors of a plurality of single-source domain teacher models;
one labeled source domain sample set and one unlabeled target domain sample set are formed into a branch, and in each branch, the feature extractor N_{f} corresponding to the domain-specific teacher model is used to extract features from the respective source domain sample and target domain sample, and the features are extracted from an original feature space into an embedded space;
embedded features are aligned at a domain level based on the maximum mean discrepancy in the feature space; the embedded features are aligned at a data pair level based on the pseudo-label triplet loss of margin-based sampling;
by minimizing the maximum mean discrepancy and the pseudo-label triplet loss, the feature extractors N_{f} of the plurality of single-source domain teacher models are trained to extract domain-invariant features in the source domain and the target domain;
S3-4: training label classifiers N_{y} of the plurality of single-source domain teacher models
in each single-source domain teacher model, the extracted source domain feature information is passed through the label classifier N_{y} to obtain predicted emotion Ŷ^{S}, and a cross-entropy between the predicted emotion Ŷ^{S} and a corresponding label Y^{S} in an actual sample is calculated; similarly, a cross-entropy between a predicted emotion Ŷ^{T} of the target domain feature information and a generated pseudo-label Ŷ^{T} is calculated;
by minimizing the two obtained cross-entropies, the label classifiers N_{y} of the plurality of single-source domain teacher models are trained to have good emotion classification ability in their respective source domains and respective target domains;
S3-5: merging knowledge of the plurality of single-source domain teacher models;
S3-6: teaching merged knowledge of the teacher models to the student model.

4. The multi-source domain adaptive EEG emotional state classification method based on knowledge distillation according to claim 2, wherein construction and training of a multi-source domain knowledge transfer framework (MS-KTF) model during implementation of the method are as follows:
S3-1: initialization;
the MS-KFT model consists of two parts: teacher models, which each are based on a corresponding single source domain, and a student model acting on a target domain. both the teacher models and the student model each consist of two modules: a domain-specific feature extractor N_{f} and a label classifier N_{y}; a plurality of single-source domain teacher models based on the multi-source domain and parameters of a target domain student are initialized;
S3-2: pre-training the plurality of single-source domain teacher models;
based on a multi-source domain sample set, a feature extractor N_{f} and a label classifier N_{y} of each domain-specific teacher model are pre-trained using a corresponding labeled single-source domain sample set, such that each domain-specific teacher model has a certain pattern recognition ability in its respective source domain;
S3-3: performing domain adaptation on feature extractors of a plurality of single-source domain teacher models;
one labeled source domain sample set and one unlabeled target domain sample set are formed into a branch, and in each branch, the feature extractor N_{f} corresponding to the domain-specific teacher model is used to extract features from the respective source domain sample and target domain sample, and the features are extracted from an original feature space into an embedded space;
embedded features are aligned at a domain level based on the maximum mean discrepancy in the feature space; the embedded features are aligned at a data pair level based on the pseudo-label triplet loss of margin-based sampling;
by minimizing the maximum mean discrepancy and the pseudo-label triplet loss, the feature extractors N_{f} of the plurality of single-source domain teacher models are trained to extract domain-invariant features in the source domain and the target domain;
S3-4: training label classifiers N_{y} of the plurality of single-source domain teacher models
in each single-source domain teacher model, the extracted source domain feature information is passed through the label classifier N_{y} to obtain predicted emotion Ŷ^{S}, and a cross-entropy between the predicted emotion Ŷ^{S} and a corresponding label Y^{S} in an actual sample is calculated; similarly, a cross-entropy between a predicted emotion Ŷ^{T} of the target domain feature information and a generated pseudo-label Ŷ^{T} is calculated;
by minimizing the two obtained cross-entropies, the label classifiers N_{y} of the plurality of single-source domain teacher models are trained to have good emotion classification ability in their respective source domains and respective target domains;
S3-5: merging knowledge of the plurality of single-source domain teacher models;
S3-6: teaching merged knowledge of the teacher models to the student model.

5. The multi-source domain adaptive EEG emotional state classification method based on knowledge distillation according to claim 3, wherein two different merging strategies are used to balance performances of the teacher models:
(1) screening the teacher models to be merged based on a voting manner;
this strategy is more suitable for a case in which teacher models have poor performance balance; based on an emotion prediction result Ŷ_{teacher}^{T} obtained by the feature extractor and the label classifier of each teacher model, with a unlabeled target domain sample a corresponding one-hot coding result Ô_{teacher}^{T} is generated; voting is performed based on the one-hot coding result Ô_{teacher}^{T} generated by each teacher model, and the voting result is deemed as a decision variable DT; in a case that the emotion prediction result Ŷ_{teacher}^{T} of the teacher model is the same as the decision variable \hat{D}^{T}, the teacher model is selected for knowledge merging;
a mean value Ŷ_{teacher}^{T} of emotion prediction results of all selected teacher models is calculated as the merged knowledge of all the teacher models;
(2) merging the teacher models in an average manner;
this strategy is more suitable for a case in which teacher models have strong performance balance; in this case, all the teacher models have a same weight, and the mean value Ŷ_{teacher}^{T} of the prediction results of all the selected teacher models is calculated as the merged knowledge of all the teacher models.

6. The multi-source domain adaptive EEG emotional state classification method based on knowledge distillation according to claim 4, wherein two different merging strategies are used to balance performances of the teacher models:
(1) screening the teacher models to be merged based on a voting manner;
this strategy is more suitable for a case in which teacher models have poor performance balance; based on an emotion prediction result Ŷ_{teacher}^{T} obtained by the feature extractor and the label classifier of each teacher model, with a unlabeled target domain sample a corresponding one-hot coding result Ô_{teacher}^{T} is generated; voting is performed based on the one-hot coding result Ô_{teacher}^{T} generated by each teacher model, and the voting result is deemed as a decision variable \hat{D}^{T}; in a case that the emotion prediction result Ŷteacher^{T} of the teacher model is the same as the decision variable \hat{D}^{T}, the teacher model is selected for knowledge merging;
a mean value Y̅_{teacher}^{T} of emotion prediction results of all selected teacher models is calculated as the merged knowledge of all the teacher models;
(2) merging the teacher models in an average manner;
this strategy is more suitable for a case in which teacher models have strong performance balance; in this case, all the teacher models have a same weight, and the mean value Y̅_{teacher}^{T} of the prediction results of all the selected teacher models is calculated as the merged knowledge of all the teacher models.

7. The multi-source domain adaptive EEG emotional state classification method based on knowledge distillation according to claim 3, wherein the teaching merged knowledge of the teacher models to the student model is as follows:
with unlabeled target domain sample data, obtaining a prediction result Ŷ_{student}^{T} of the student model through the feature extractor and the label classifier of the student model;
based on a predetermined distillation temperature, performing smoothing processing on the merged knowledge Y̅_{teacher}^{T} of the teacher models and the prediction result Ŷ_{student}^{T} of the student model; and using Kullback-Leibler (KL) divergence to evaluate a difference between two prediction results;
by minimizing the KL divergence between the teacher models and the student model so that the student model learns the knowledge of the teacher models, obtaining more extensive feature extraction and label classification ability than the teacher models in the target domain.

8. The multi-source domain adaptive EEG emotional state classification method based on knowledge distillation according to claim 4, wherein the teaching merged knowledge of the teacher models to the student model is as follows:
with unlabeled target domain sample data, obtaining a prediction result Ŷ_{student}^{T} of the student model through the feature extractor and the label classifier of the student model;
based on a predetermined distillation temperature, performing smoothing processing on the merged knowledge Y̅_{teacher}^{T} of the teacher models and the prediction result Ŷ_{student}^{T} of the student model; and using Kullback-Leibler (KL) divergence to evaluate a difference between two prediction results;
by minimizing the KL divergence between the teacher models and the student model so that the student model learns the knowledge of the teacher models, obtaining more extensive feature extraction and label classification ability than the teacher models in the target domain.

9. The multi-source domain adaptive EEG emotional state classification method based on knowledge distillation according to claim 3, wherein the initialization is implemented as follows:
S3-1-1: data division of the model, in which specific division is described as follows:
for the cross-subject scenario: a target domain sample set of the model is U^{T}={Xᵢ}, where Xᵢ indicates a feature data set of an i-th subject; Xᵢ={xⱼ}ⱼ₌₁ⁿ, xⱼ indicates a j-th sample in Xᵢ, and n indicates a number of samples in Xᵢ; a multi-source domain sample set of the model is
$${U}^{s} = {\{ {( {X}_{i} , {Y}_{i} )}_{i ∈ {P}_{j}} \}}_{j = 1}^{⌊ \frac{N - 1}{R} ⌋} ,$$
Pⱼ⊆[N]\i, Pⱼ∩Pₖ=Ø, ∀i∀k, i≠j, where [N]\i indicates a serial number set of all the subjects after i-th subject data is removed, and Pⱼ indicates a serial number set of the subjects included in a j-th source domain;
for the cross-session scenario: the target domain sample set of the model is U^{T}={(Xᵢ)ᵢ₌₁^{N}}ⱼ, where Xᵢ indicates a feature data set of an i-th subject; j indicates a j-th session (period); the multi-source domain sample set of the model is U^{S}={(Xᵢ,Yᵢ)ᵢ₌₁^{N}}ₖ, k∈[D]/j, where [D]/j indicates a serial number set of all the sessions after a j-th session is removed;
S3-1-2: data input of the model
one labeled source domain sample set U^{S} and one unlabeled target domain sample set U^{T} are formed into a branch for a subsequent training of each single-source domain teacher model; and for the student model, only the unlabeled target domain sample set U^{T} is used.

10. The multi-source domain adaptive EEG emotional state classification method based on knowledge distillation according to claim 4, wherein the initialization is implemented as follows:
S3-1-1: data division of the model, in which specific division is described as follows:
for the cross-subject scenario: a target domain sample set of the model is U^{T}={Xᵢ}, where Xᵢ indicates a feature data set of an i-th subject; Xᵢ={xⱼ}ⱼ₌₁ⁿ, xⱼ indicates a j-th sample in Xᵢ, and n indicates a number of samples in Xᵢ; a multi-source domain sample set of the model is
$${U}^{s} = {\{ {( {X}_{i} , {Y}_{i} )}_{i ∈ {P}_{j}} \}}_{j = 1}^{⌊ \frac{N - 1}{R} ⌋} ,$$
Pⱼ⊆[N]\i, Pⱼ∩Pₖ=Ø, ∀i∀k, i≠j, where [N]\i indicates a serial number set of all the subjects after i-th subject data is removed, and Pⱼ indicates a serial number set of the subjects included in a j-th source domain;
for the cross-session scenario: the target domain sample set of the model is U^{T}={(Xᵢ)ᵢ₌₁^{N}}ⱼ, where Xᵢ indicates a feature data set of an i-th subject; j indicates a j-th session (period); the multi-source domain sample set of the model is U^{S}={(Xᵢ,Yᵢ)ᵢ₌₁^{N}}ₖ, k∈[D]/j, where [D]/j indicates a serial number set of all the sessions after a j-th session is removed;
S3-1-2: data input of the model
one labeled source domain sample set U^{S} and one unlabeled target domain sample set U^{T} are formed into a branch for a subsequent training of each single-source domain teacher model;
and for the student model, only the unlabeled target domain sample set U^{T} is used.

11. The multi-source domain adaptive EEG emotional state classification method based on knowledge distillation according to claim 5, wherein the initialization is implemented as follows:
S3-1-1: data division of the model, in which specific division is described as follows:
for the cross-subject scenario: a target domain sample set of the model is U^{T}={Xᵢ}, where Xᵢ indicates a feature data set of an i-th subject; Xᵢ={xⱼ}ⱼ₌₁ⁿ, xⱼ indicates a j-th sample in Xᵢ, and n indicates a number of samples in Xᵢ; a multi-source domain sample set of the model is
$${U}^{s} = {\{ {( {X}_{i} , {Y}_{i} )}_{i ∈ {P}_{j}} \}}_{j = 1}^{⌊ \frac{N - 1}{R} ⌋} ,$$
Pⱼ⊆[N]\i, Pⱼ∩Pₖ=Ø, ∀i∀k, i≠j, where [N]\i indicates a serial number set of all the subjects after i-th subject data is removed, and Pⱼ indicates a serial number set of the subjects included in a j-th source domain;
for the cross-session scenario: the target domain sample set of the model is U^{T}={(Xᵢ)ᵢ₌₁^{N}}ⱼ, where Xᵢ indicates a feature data set of an i-th subject; j indicates a j-th session (period); the multi-source domain sample set of the model is U^{S}={(Xᵢ,Yᵢ)ᵢ₌₁^{N}}ₖ, k∈[D]/j, where [D]/j indicates a serial number set of all the sessions after a j-th session is removed;
S3-1-2: data input of the model
one labeled source domain sample set U^{S} and one unlabeled target domain sample set U^{T} are formed into a branch for a subsequent training of each single-source domain teacher model; and for the student model, only the unlabeled target domain sample set U^{T} is used.

12. The multi-source domain adaptive EEG emotional state classification method based on knowledge distillation according to claim 6, wherein the initialization is implemented as follows:
S3-1-1: data division of the model, in which specific division is described as follows:
for the cross-subject scenario: a target domain sample set of the model is U^{T}={Xᵢ}, where Xᵢ indicates a feature data set of an i-th subject; Xᵢ={xⱼ}ⱼ₌₁ⁿ, xⱼ indicates a j-th sample in Xᵢ, and n indicates a number of samples in Xᵢ; a multi-source domain sample set of the model is
$${U}^{s} = {\{ {( {X}_{i} , {Y}_{i} )}_{i ∈ {P}_{j}} \}}_{j = 1}^{⌊ \frac{N - 1}{R} ⌋} ,$$
Pⱼ⊆[N]\i, Pⱼ∩Pₖ=Ø, ∀i∀k, i≠j, where [N]\i indicates a serial number set of all the subjects after i-th subject data is removed, and Pⱼ indicates a serial number set of the subjects included in a j-th source domain;
for the cross-session scenario: the target domain sample set of the model is U^{T}={(Xᵢ)ᵢ₌₁^{N}}ⱼ, where Xᵢ indicates a feature data set of an i-th subject; j indicates a j-th session (period); the multi-source domain sample set of the model is U^{S}={(Xᵢ,Yᵢ)ᵢ₌₁^{N}}ₖ, k∈[D]/j, where [D]/j indicates a serial number set of all the sessions after a j-th session is removed;
S3-1-2: data input of the model
one labeled source domain sample set U^{S} and one unlabeled target domain sample set U^{T} are formed into a branch for a subsequent training of each single-source domain teacher model; and for the student model, only the unlabeled target domain sample set U^{T} is used.

13. The multi-source domain adaptive EEG emotional state classification method based on knowledge distillation according to claim 7, wherein the initialization is implemented as follows:
S3-1-1: data division of the model, in which specific division is described as follows:
for the cross-subject scenario: a target domain sample set of the model is U^{T}={Xᵢ}, where Xᵢ indicates a feature data set of an i-th subject; Xᵢ={xⱼ}ⱼ₌₁ⁿ, xⱼ indicates a j-th sample in Xᵢ, and n indicates a number of samples in Xᵢ; a multi-source domain sample set of the model is
$${U}^{s} = {\{ {( {X}_{i} , {Y}_{i} )}_{i ∈ {P}_{j}} \}}_{j = 1}^{⌊ \frac{N - 1}{R} ⌋} ,$$
Pⱼ⊆[N]\i, Pⱼ∩Pₖ=Ø, ∀i∀k, i≠j, where [N]\i indicates a serial number set of all the subjects after i-th subject data is removed, and Pⱼ indicates a serial number set of the subjects included in a j-th source domain;
for the cross-session scenario: the target domain sample set of the model is U^{T}={(Xᵢ)ᵢ₌₁^{N}}ⱼ, where Xᵢ indicates a feature data set of an i-th subject; j indicates a j-th session (period); the multi-source domain sample set of the model is U^{S}={(Xᵢ,Yᵢ)ᵢ₌₁^{N}}ₖ, k∈[D]/j, where [D]/j indicates a serial number set of all the sessions after a j-th session is removed;
S3-1-2: data input of the model
one labeled source domain sample set U^{S} and one unlabeled target domain sample set U^{T} are formed into a branch for a subsequent training of each single-source domain teacher model; and for the student model, only the unlabeled target domain sample set U^{T} is used.

14. The multi-source domain adaptive EEG emotional state classification method based on knowledge distillation according to claim 8, wherein the initialization is implemented as follows:
S3-1-1: data division of the model, in which specific division is described as follows:
for the cross-subject scenario: a target domain sample set of the model is U^{T}={Xᵢ}, where Xᵢ indicates a feature data set of an i-th subject; Xᵢ={xⱼ}ⱼ₌₁ⁿ, xⱼ indicates a j-th sample in Xᵢ, and n indicates a number of samples in Xᵢ; a multi-source domain sample set of the model is
$${U}^{s} = {\{ {( {X}_{i} , {Y}_{i} )}_{i ∈ {P}_{j}} \}}_{j = 1}^{⌊ \frac{N - 1}{R} ⌋} ,$$
Pⱼ⊆[N]\i, Pⱼ∩Pₖ=Ø, ∀i∀k, i≠j, where [N]\i indicates a serial number set of all the subjects after i-th subject data is removed, and Pⱼ indicates a serial number set of the subjects included in a j-th source domain;
for the cross-session scenario: the target domain sample set of the model is U^{T}={(Xᵢ)ᵢ₌₁^{N}}ⱼ, where Xᵢ indicates a feature data set of an i-th subject; j indicates a j-th session (period); the multi-source domain sample set of the model is U^{S}={(Xᵢ,Yᵢ)ᵢ₌₁^{N}}ₖ, k∈[D]/j, where [D]/j indicates a serial number set of all the sessions after a j-th session is removed;
S3-1-2: data input of the model
one labeled source domain sample set U^{S} and one unlabeled target domain sample set U^{T} are formed into a branch for a subsequent training of each single-source domain teacher model; for the student model, only the unlabeled target domain sample set U^{T} is used.

15. The multi-source domain adaptive EEG emotional state classification method based on knowledge distillation according to claim 3, wherein the performing domain adaptation on the feature extractors of the plurality of single-source domain teacher models is as follows:
after passing through the feature extractor N_{f} of the domain-specific teacher model, respective low-dimensional features F^{S} and F^{T} of the corresponding source domain data U^{S} and the target domain data U^{T} are extracted; in order to ensure unbiased adaptability of the extracted features, two methods comprising a domain-level distribution alignment and a data-pair-level distribution alignment are used;
S3-4-1: domain-level distribution alignment
based on the pseudo-label triple loss and the maximum mean discrepancy, the domain adaptation of each teacher model is performed; in a training process, a distance between the source domain and the target domain in the feature space is reduced by minimizing a maximum mean discrepancy (MMD) loss, so as to achieve the domain-level alignment, and a specific formula is as follows:
$$M M {D}_{( {F}^{S} , {F}^{T} )} = {‖ \frac{1}{{N}^{S}} {\sum }_{i = 1}^{{N}^{S}} {F}_{i}^{S} - \frac{1}{{N}^{T}} {\sum }_{j = 1}^{{N}^{T}} {F}_{j}^{T} ‖}_{ℋ}^{2} \qquad ( 1 )$$
where Fᵢ^{S} and Fⱼ^{T} indicate extracted low-dimensional features of the i-th sample in the source domain and the j-th sample in the target domain, respectively; N^{S} and N^{T} indicate a number of source domain samples and a number of target domain samples respectively;
S3-4-2: data-pair-level distribution alignment
the triple loss of margin-based sampling is used to perform the data-pair-level distribution alignment, and a margin-based score of the prediction result of each sample is used as a basis for determining whether the sample is sampled, and this method is expressed by a following formula:
$$margin ( x ) = {{g}_{θ} ( {F}^{T} )}_{{i}^{*}} - {max}_{i ∈ [ k ] ∖ {i}^{*}} \{ {{g}_{θ} ( {F}^{T} )}_{i} \} {X}_{s e lected} = \{ {x}_{j} \vert margin ( {x}_{j} ) \geq Threshold , {x}_{j} ∈ X \} \qquad ( 3 )$$
where x is an input sample, g_{θ} is an abstract function of the label classifier, i* is a category with a highest prediction probability in the prediction result, k is a number of all categories, [k]\i* indicates a set of all the categories except i*, and Threshold is a predetermined threshold of margin-based sampling;
the triplet loss requires sampling in a form of triplet {xᵢ^{a},xᵢ^{p},xᵢⁿ}^{N}^{ₜᵣᵢₚ}, in which an anchor sample xᵢ^{a} and a positive sample xᵢ^{p} are different samples of a same category from a i-th triplet, xᵢⁿ is any sample of the i-th triplet of a category different from that of anchor sample xᵢ^{a}; a purpose of the triple loss is to ensure that a distance between embedded features of a positive sample pair (xᵢ^{a},xᵢ^{p}) plus a fixed margin value is smaller than a distance between embedded features of a negative sample pair (xᵢ^{a},xᵢⁿ); for a mini-batch sample set, the triple loss is defined as:
$${d}_{p} ( {x}_{i}^{a} , {x}_{i}^{p} ) = ‖ {f}_{θ} ( {x}_{i}^{a} ) - {f}_{θ} ( {x}_{i}^{p} ) ‖ {d}_{n} ( {x}_{i}^{a} , {x}_{i}^{n} ) = ‖ {f}_{θ} ( {x}_{i}^{a} ) - {f}_{θ} ( {x}_{i}^{n} ) ‖ {ℒ}_{trip} = \frac{1}{N} {\sum }_{{x}_{i} ∈ {X}_{s e l e c t e d}} max \{ {d}_{p} ( {x}_{i}^{a} , {x}_{i}^{p} ) - {d}_{n} ( {x}_{i}^{a} , {x}_{i}^{n} ) + α , 0 \} \qquad ( 4 )$$
where N is a number of samples contained in X_{selected}, α is a predetermined margin value for guiding separability, d(⋅) is a function for calculating an Euler distance between regularized embedded feature pairs, and f_{θ}(⋅) is an abstract function for feature extraction.

16. The multi-source domain adaptive EEG emotional state classification method based on knowledge distillation according to claim 4, wherein the performing domain adaptation on the feature extractors of the plurality of single-source domain teacher models is as follows:
after passing through the feature extractor N_{f} of the domain-specific teacher model, respective low-dimensional features F^{S} and F^{T} of the corresponding source domain data U^{S} and the target domain data U^{T} are extracted; in order to ensure unbiased adaptability of the extracted features, two methods comprising a domain-level distribution alignment and a data-pair-level distribution alignment are used;
S3-4-1: domain-level distribution alignment
based on the pseudo-label triple loss and the maximum mean discrepancy, the domain adaptation of each teacher model is performed; in a training process, a distance between the source domain and the target domain in the feature space is reduced by minimizing a maximum mean discrepancy (MMD) loss, so as to achieve the domain-level alignment, and a specific formula is as follows:
$$MM {D}_{( {F}^{S} , {F}^{T} )} = {‖ \frac{1}{{N}^{S}} {\sum }_{i = 1}^{{N}^{S}} {F}_{i}^{S} - \frac{1}{{N}^{T}} {\sum }_{j = 1}^{{N}^{T}} {F}_{j}^{T} ‖}_{ℋ}^{2} \qquad ( 1 )$$
where Fᵢ^{S} and Fⱼ^{T} indicate extracted low-dimensional features of the i-th sample in the source domain and the j-th sample in the target domain, respectively; N^{S} and N^{T} indicate a number of source domain samples and a number of target domain samples respectively;
S3-4-2: data-pair-level distribution alignment
the triple loss of margin-based sampling is used to perform the data-pair-level distribution alignment, and a margin-based score of the prediction result of each sample is used as a basis for determining whether the sample is sampled, and this method is expressed by a following formula:
$$margin ( x ) = {{g}_{θ} ( {F}^{T} )}_{{i}^{*}} - {max}_{i ∈ [ k ] ∖ {i}^{*}} \{ {{g}_{θ} ( {F}^{T} )}_{i} \} {X}_{s e lected} = \{ {x}_{j} \vert margin ( {x}_{j} ) \geq Threshold , {x}_{j} ∈ X \} \qquad ( 3 )$$
where x is an input sample, g_{θ} is an abstract function of the label classifier, i* is a category with a highest prediction probability in the prediction result, k is a number of all categories, [k]\i* indicates a set of all the categories except i*, and Threshold is a predetermined threshold of margin-based sampling;
the triplet loss requires sampling in a form of triplet {xᵢ^{a},xᵢ^{p},xᵢⁿ}^{N}^{ₜᵣᵢₚ}, in which an anchor sample xᵢ^{a} and a positive sample xᵢ^{p} are different samples of a same category from a i-th triplet, xᵢⁿ is any sample of the i-th triplet of a category different from that of anchor sample xᵢ^{a}; a purpose of the triple loss is to ensure that a distance between embedded features of a positive sample pair (xᵢ^{a},xᵢ^{p}) plus a fixed margin value is smaller than a distance between embedded features of a negative sample pair (xᵢ^{a},xᵢⁿ); for a mini-batch sample set, the triple loss is defined as:
$${d}_{p} ( {x}_{i}^{a} , {x}_{i}^{p} ) = ‖ {f}_{θ} ( {x}_{i}^{a} ) - {f}_{θ} ( {x}_{i}^{p} ) ‖ {d}_{n} ( {x}_{i}^{a} , {x}_{i}^{n} ) = ‖ {f}_{θ} ( {x}_{i}^{a} ) - {f}_{θ} ( {x}_{i}^{n} ) ‖ {ℒ}_{trip} = \frac{1}{N} {\sum }_{{x}_{i} ∈ {X}_{s e l e c t e d}} max \{ {d}_{p} ( {x}_{i}^{a} , {x}_{i}^{p} ) - {d}_{n} ( {x}_{i}^{a} , {x}_{i}^{n} ) + α , 0 \} \qquad ( 4 )$$
where N is a number of samples contained in X_{selected}, α is a predetermined margin value for guiding separability, d(⋅) is a function for calculating an Euler distance between regularized embedded feature pairs, and f_{θ}(⋅) is an abstract function for feature extraction.

17. The multi-source domain adaptive EEG emotional state classification method based on knowledge distillation according to claim 5, wherein the performing domain adaptation on the feature extractors of the plurality of single-source domain teacher models is as follows:
after passing through the feature extractor N_{f} of the domain-specific teacher model, respective low-dimensional features F^{S} and F^{T} of the corresponding source domain data U^{S} and the target domain data U^{T} are extracted; in order to ensure unbiased adaptability of the extracted features, two methods comprising a domain-level distribution alignment and a data-pair-level distribution alignment are used;
S3-4-1: domain-level distribution alignment
based on the pseudo-label triple loss and the maximum mean discrepancy, the domain adaptation of each teacher model is performed; in a training process, a distance between the source domain and the target domain in the feature space is reduced by minimizing a maximum mean discrepancy (MMD) loss, so as to achieve the domain-level alignment, and a specific formula is as follows:
$$MM {D}_{( {F}^{S} , {F}^{T} )} = {‖ \frac{1}{{N}^{S}} {\sum }_{i = 1}^{{N}^{S}} {F}_{i}^{S} - \frac{1}{{N}^{T}} {\sum }_{j = 1}^{{N}^{T}} {F}_{j}^{T} ‖}_{ℋ}^{2} \qquad ( 1 )$$
where Fᵢ^{S} and Fⱼ^{T} indicate extracted low-dimensional features of the i-th sample in the source domain and the j-th sample in the target domain, respectively; N^{S} and N^{T} indicate a number of source domain samples and a number of target domain samples respectively;
S3-4-2: data-pair-level distribution alignment
the triple loss of margin-based sampling is used to perform the data-pair-level distribution alignment, and a margin-based score of the prediction result of each sample is used as a basis for determining whether the sample is sampled, and this method is expressed by a following formula:
$$margin ( x ) = {{g}_{θ} ( {F}^{T} )}_{{i}^{*}} - {max}_{i ∈ [ k ] ∖ {i}^{*}} \{ {{g}_{θ} ( {F}^{T} )}_{i} \} {X}_{s e lected} = \{ {x}_{j} \vert margin ( {x}_{j} ) \geq Threshold , {x}_{j} ∈ X \} \qquad ( 3 )$$
where x is an input sample, g_{θ} is an abstract function of the label classifier, i* is a category with a highest prediction probability in the prediction result, k is a number of all categories, [k]\i* indicates a set of all the categories except i*, and Threshold is a predetermined threshold of margin-based sampling;
the triplet loss requires sampling in a form of triplet {xᵢ^{a},xᵢ^{p},xᵢⁿ}^{N}^{ₜᵣᵢₚ}, in which an anchor sample xᵢ^{a} and a positive sample xᵢ^{p} are different samples of a same category from a i-th triplet, xᵢⁿ is any sample of the i-th triplet of a category different from that of anchor sample xᵢ^{a}; a purpose of the triple loss is to ensure that a distance between embedded features of a positive sample pair (xᵢ^{a},xᵢ^{p}) plus a fixed margin value is smaller than a distance between embedded features of a negative sample pair (xᵢ^{a},xᵢⁿ); for a mini-batch sample set, the triple loss is defined as:
$${d}_{p} ( {x}_{i}^{a} , {x}_{i}^{p} ) = ‖ {f}_{θ} ( {x}_{i}^{a} ) - {f}_{θ} ( {x}_{i}^{p} ) ‖ {d}_{n} ( {x}_{i}^{a} , {x}_{i}^{n} ) = ‖ {f}_{θ} ( {x}_{i}^{a} ) - {f}_{θ} ( {x}_{i}^{n} ) ‖ {ℒ}_{trip} = \frac{1}{N} {\sum }_{{x}_{i} ∈ {X}_{s e l e c t e d}} max \{ {d}_{p} ( {x}_{i}^{a} , {x}_{i}^{p} ) - {d}_{n} ( {x}_{i}^{a} , {x}_{i}^{n} ) + α , 0 \} \qquad ( 4 )$$
where N is a number of samples contained in X_{selected}, α is a predetermined margin value for guiding separability, d(⋅) is a function for calculating an Euler distance between regularized embedded feature pairs, and f_{θ}(⋅) is an abstract function for feature extraction.

18. The multi-source domain adaptive EEG emotional state classification method based on knowledge distillation according to claim 3, wherein the cross-entropy loss is used as an evaluation index of a classification result of the label classifier in the source domain and the target domain, a source classification loss (SCL) is used as a classification loss in the source domain, and a target classification loss (TCL) is used as a classification loss in the target domain;
in the source domain, there is a real label, so that the SCL uses the real label and the classification result of the label classifier as comparison objects, and a specific formula is as follows:
$${w}_{i} = \{ \begin{matrix}1 , if {\hat{y}}_{i}^{s} = {y}_{i}^{s} \\ 0 , otherwise\end{matrix} {ℒ}_{S C L} = - \sum {w}_{i} ln ( {g}_{θ} ( {f}_{θ} ( {x}_{i} ) ) ) , \qquad ( 5 )$$
where xᵢ is an i-th source domain input sample, yᵢ^{S} is a real label of the i-th source domain input sample, ŷᵢ^{S} is a prediction result of the label classifier for the i-th source domain input sample, f_{θ}(⋅) is an abstract function for feature extraction, and g_{θ}(⋅) is an abstract function of the label classifier;
in the target domain, the sample lacks a real label, and the corresponding TCL uses a generated pseudo label and the classification result of the label classifier as comparison objects, and a specific formula is as follows:
$${\tilde{y}}_{i}^{T} = arg max ( {g}_{θ} ( {f}_{θ} ( {x}_{i} ) ) ) , {w}_{i} = \{ \begin{matrix}1 , if {\hat{y}}_{i}^{T} = {y}_{i}^{T} \\ 0 , otherwise\end{matrix} , {ℒ}_{S C L} = - \sum {w}_{i} ln ( {g}_{θ} ( {f}_{θ} ( {x}_{i} ) ) ) , \qquad ( 6 )$$
where xᵢ is an i-th target domain input sample, \tilde{y}ᵢ^{T} is a pseudo label generated by the i-th target domain input sample, ŷᵢ^{T} is a prediction result of the label classifier for the i-th target domain input sample, f_{θ}(⋅) is an abstract function for feature extraction, and g_{θ}(⋅) is an abstract function of the label classifier.

19. The multi-source domain adaptive EEG emotional state classification method based on knowledge distillation according to claim 3, wherein a goal optimization and training of the single-source domain teacher model are as follows:
in a domain adaptation stage of the teacher model, a final optimization goal is shown in a following formula:
$${min}_{{f}_{θ} , {g}_{θ}} {ℒ}_{D} = {min}_{{f}_{θ} , {g}_{θ}} ( {ℒ}_{S C L} + {βℒ}_{M M D} + {γℒ}_{trip} + {σℒ}_{T C L} ) \qquad ( 7 )$$
where β, γ and σ are weighting factors for balancing a loss function;
by using a random gradient optimizer and combining with a mini-batch training mode, domain invariant features are obtained for each pair of source domain and target domain at the domain level and a data pair level through minimizing the MMD loss and the triple loss ( [symbol] _{MMD}, [symbol] ₜᵣᵢₚ) in formula (7); by minimizing the classification losses ( [symbol] _{SCL}, [symbol] _{TCL}) in the source domain and the target domain, a better classifier is obtained, which accurately predicts source domain samples without sacrificing an ability to discriminate the target domain samples.

20. The multi-source domain adaptive EEG emotional state classification method based on knowledge distillation according to claim 3, wherein a training of the student model is as follows:
after domain adaptation of the teacher models, a plurality of single-source domain models are obtained, in order to better merge the knowledge of the teacher models, a voting-based method is used to select the knowledge of the teacher models to be merged, expressed as a following formula:
$$\{ {\tilde{y}}_{i} \} = {mode}_{j = 1}^{{N}_{t}} ( {\hat{y}}_{i}^{{T}_{j}} ) , {mask}_{i}^{{T}_{j}} = \{ \begin{matrix}0 , if {\hat{y}}_{i}^{{T}_{j}} ∉ \{ {\tilde{y}}_{i} \} \\ 1 , otherwise\end{matrix} , {merge}_{i} = {avg}_{j = 1}^{{N}_{t}} ( {g}_{θ}^{{T}_{j}} ( {f}_{θ}^{{T}_{j}} ( {x}_{i} ) ) * {mask}_{i}^{{T}_{j}} ) \qquad ( 8 )$$
where xᵢ is an i-th input sample, Nₜ is a number of teacher models, mode(⋅) is a function for finding a mode/multiple modes, * is a point multiplication function, ŷᵢ^{T}^{ⱼ} is a prediction result of a j-th teacher model for the i-th input sample, and {\tilde{y}ᵢ} is a decision label set for generating a teacher model mask of the i-th input sample;
after obtaining the merged knowledge of the plurality of single-source domain teacher models, KL divergence is used to evaluate a difference between the prediction result of the teacher models and the prediction result of the student model, and the formula is as follows:
$${ℒ}_{K D} = KLD [ ( {g}_{θ} ( {f}_{θ} ( X ) ) ; T ) , ( merge ; T ) ] \qquad ( 9 )$$
where X is an input sample set, merge is a merged teacher knowledge set, T is a predetermined temperature coefficient for controlling a smoothness of softmax function, and KLD[p,q] is an evaluation function for measuring a KL divergence between a distribution p and a distribution q;
by using an Adam optimizer and combining with a mini-batch training mode, a KL loss in the formula (9) is minimized, so that the student model fully learns the merged knowledge of the teacher model and obtains better performance in the target domain.
